(19) United States
(12) Patent Application Publication
(10) Pub. No.: US 2019/0057547 A1
(43) Pub. Date: Feb. 21, 2019

(54) System and Method for Imaging a Mouth in Real Time During a Dental Procedure

(71) Applicant: James A. Abraham, II, Park City, UT (US)

(72) Inventor: James A. Abraham, II, Park City, UT (US)

(21) Appl. No.: 15/678,276

(22) Filed: Aug. 16, 2017

(51) Int. Cl. G06T 19/00; H04N 5/225; G06K 9/00; H04N 5/232; A61B 1/24; A61B 1/00; A41D 19/00; A61C 9/00

(52) U.S. Cl. CPC G06T 19/003; H04N 5/2253; G06K 9/00248; H04N 5/23206; H04N 2005/2255; A61B 1/00147; A41D 19/0027; A61B 1/00045; A61C 9/0053; A61B 1/24

(57) ABSTRACT

A dental imaging system for imaging a mouth in real time during a dental procedure includes a signal generator that selectively transmits one or more signals into the mouth and a signal receiver for receiving the transmitted signals. The dental imaging system includes a computing system in data communication with the signal generator and in data communication with the signal receiver so as to receive signal data from the signal receiver, the computing system being configured to process the received signal data to generate a 3-dimensional model (“3D model”), the 3D model having a 3D model visualization component. A display screen is in data communication with the computing system that is configured to display the 3D visualization component of the 3D model. The signal transceivers and display screen may be mounted to a glove worn by the dentist.

### BACKGROUND OF THE INVENTION

This invention relates generally to imaging systems and, more particularly, to a mouth imaging system that provides a real time video image of a mouth and structure therein so that a doctor or technician has current visualization of all structures of an interior mouth area of a patient in real time as a dental or orthodontic procedure is being executed.

Traditionally, an X-ray picture or image is taken of a portion of a dental patient's teeth for review by a dentist, orthodontist, or surgeon before a dental procedure, implant surgery, or the like is begun. For instance, an x-ray is sufficient to identify the position of a cavity or decayed area before a dentist begins a repair procedure, i.e. to “fill the cavity.” However, many dental procedures are more complex and may involve multiple teeth, multiple dental structures, and may even take many hours. For instance, the removal of teeth or installation of tooth inserts may require images to be updated multiple times during a procedure, require current knowledge of peripheral nerves in relative position to the dental work, and the like.

X-ray images taken before the procedure may need to be updated as a procedure is executed, do not provide real time images of the patient's entire mouth, and do not provide images that stay in close proximity to the patient's mouth for easy review by the dentist performing the procedure. Radiological signals include x-ray, ultrasound, magnetic resonance, and the like.

Therefore, it would be desirable to have a non-radiological dental imaging system that provides a video image of a mouth, including imaging of teeth, implants, gums, cheeks, gums, and the like. Further, it would be desirable to have a non-radiological dental imaging system that displays a 3D model in real time according to signal data reflected from structures in a patient's mouth.

### SUMMARY OF THE INVENTION

A dental imaging system for imaging a mouth in real time during a dental procedure according to the present invention includes a signal generator that selectively transmits one or more signals into the mouth and a signal receiver for receiving the transmitted signals. The dental imaging system includes a computing system in data communication with the signal generator and in data communication with the signal receiver so as to receive signal data from the signal receiver, the computing system being configured to process the received signal data to generate a 3-dimensional model (“3D model”), the 3D model having a 3D model visualization component. A display screen is in data communication with the computing system that is configured to display the 3D visualization component of the 3D model.

Therefore, a general object of this invention is to provide a dental imaging system for generating a 3-dimensional image of a patient's mouth in real time during a dental procedure.

Another object of this invention is to provide a dental imaging system, as aforesaid, including a plurality of signal generators configured to emit non-radiological signals against mouth structures and a plurality of signal receivers for receiving the transmitted signals.

Still another object of this invention is to provide a dental imaging system, as aforesaid, which includes a glove wearable on a doctor's hand and configured to include the transmitters and receivers such that a patient's mouth may be imaged from close range as determined by the doctor wearing the glove.

Yet another object of this invention is to provide a dental imaging system, as aforesaid, that includes a computing system configured to process received signals and to generate a 3D image in real time as signals are received.

A further object of this invention is to provide a dental imaging system, as aforesaid, having a display mount such that a display screen may be coupled to the glove in close proximity to the mouth that is being scanned in real time.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a dental imaging system according to a preferred embodiment of the present invention, illustrated in use with a glove and display mount;

FIG. 2a is a top view of the dental imaging system as in FIG. 1 with the display screen coupled to the display mount in one position;

FIG. 2b is a top view of the dental imaging system as in FIG. 1 with the display screen coupled to the display mount in another position;

FIG. 3a is a front view of the dental imaging system as in FIG. 2a;

FIG. 3b is an isolated view on an enlarged basis taken from FIG. 3a;

FIG. 4a is a top view of the dental imaging system as in FIG. 2a;

FIG. 4b is a sectional view taken along line 4b-4b of FIG. 4a;

FIG. 4c is an isolated view on an enlarged basis taken from FIG. 4b;

FIG. 5 is a rear perspective view of the dental imaging system as in FIG. 1;

FIG. 6 is a bottom view of the dental imaging system as in FIG. 2a;

FIG. 7 is a diagrammatic view of the dental imaging system according to the preferred embodiment of the present invention;

FIG. 8 is a diagrammatic view of a signal generator emitting a signal against a mouth structure and said signal being reflected thereby;

FIG. 9 is a diagrammatic view of a glove having a plurality of signal generators spaced apart and coupled thereto according to the present invention;

FIG. 10 is a diagrammatic of the dental imaging system of FIG. 1 in use on both hands of a user; and

FIG. 11 is a block diagram of the dental imaging system according to the present invention.

FIG. 12a is a perspective view of a probe having a plurality of signal transmitters and receivers configured to image a patient's mouth; and

FIG. 12b is a top view of the probe as in FIG. 12b.

### DESCRIPTION OF THE PREFERRED EMBODIMENT

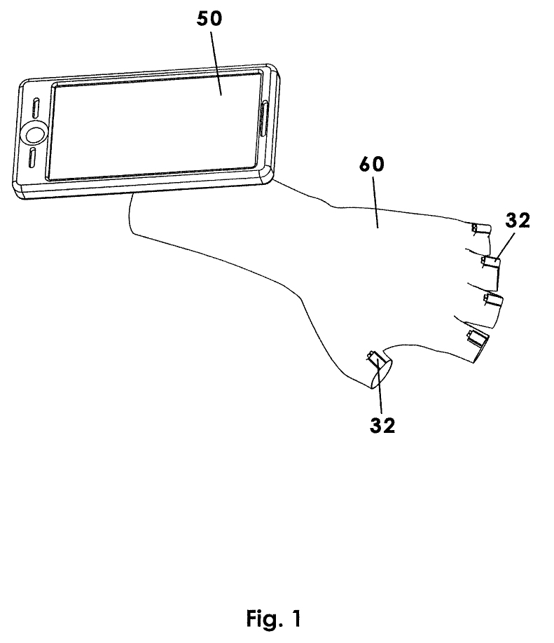

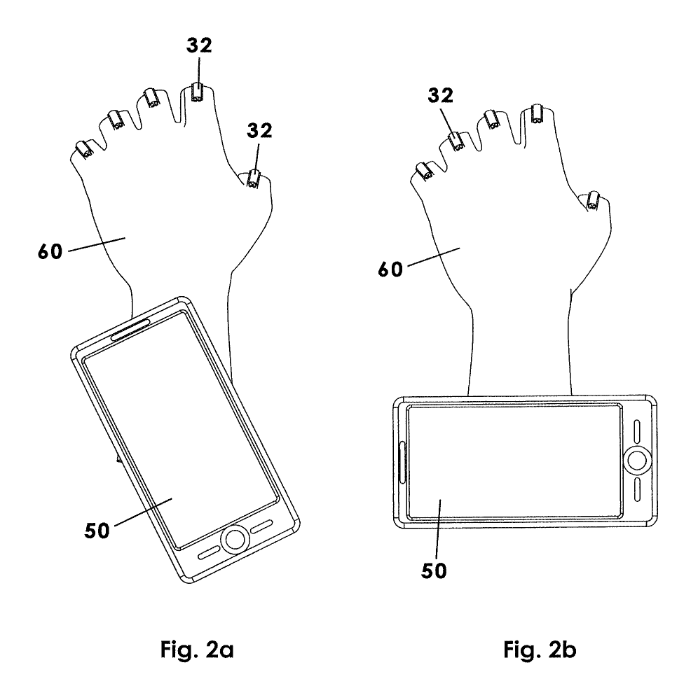

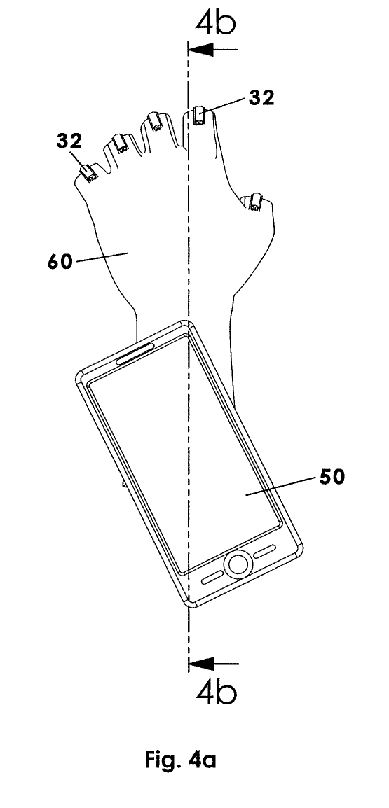

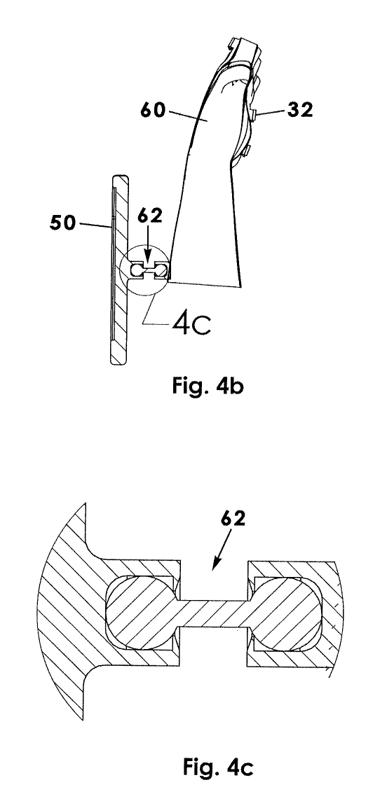

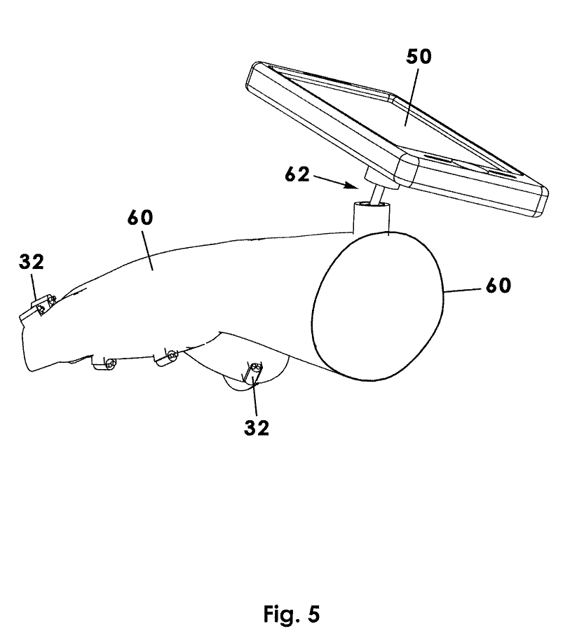

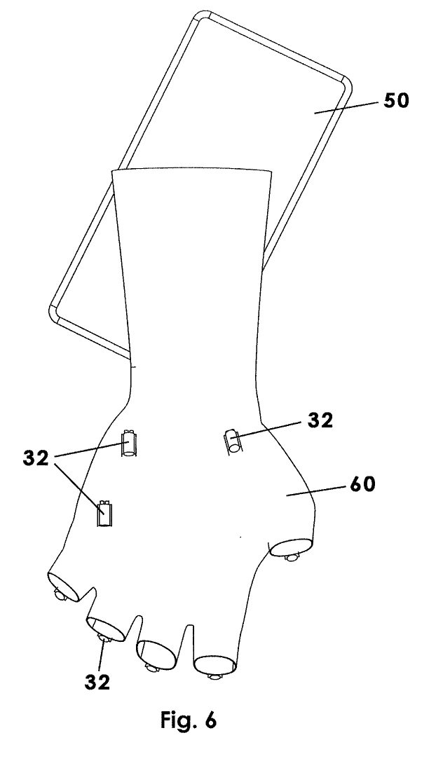

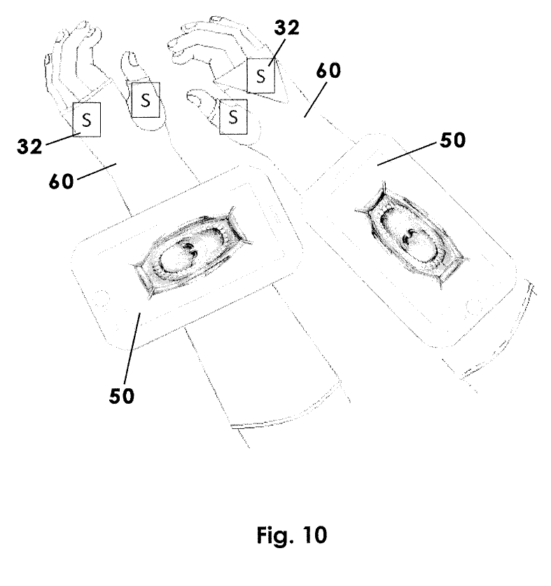

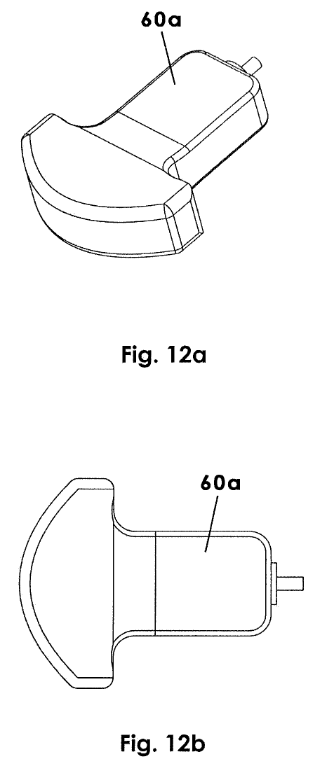

A dental imaging system and method according to a preferred embodiment of the present invention will now be described in detail with reference to FIGS. 1 to 12b of the accompanying drawings. The dental imaging system 10 includes at least one signal generator 20 and at least one signal receiver 30, a computing system in communication with the at least one signal receiver 30 for processing the received signal data and generating a 3D model of a mouth in real time.

The present technology relates to a system configured for full mouth imaging of the teeth, gums, bones, tongue, cheeks, dental fixtures, and features thereof in real time. The system includes sensors that can sense the shapes and tissue hardness/softness as well as any other parameter that can be sensed. The sensors can be part of a system that includes a signal generator 20 (also referred to as a signal transmitter) that transmits signals into the mouth and a signal receiver 30 that receives deflected signals from the mouth. The sensors can emit any kind of signal that can contact a surface and deflect therefrom and toward a signal receiver 30. However, the sensors preferred in the present invention are not part of a radiographic imaging system, and the present invention affirmatively omits radiographic signal generators, radiographic signal transmitters, or radiographic receivers. It is understood that the signal generator 20 and signal receiver 30 may actually include a plurality of signal generators and a plurality of signal receivers. Further, each signal generator/signal receiver pair may be housed in a unitary housing and be referred to as a transceiver.

In one embodiment, the dental imaging system 10 can include a vibration emitter configured to emit fluid vibrations and a sensor receiver to receive the deflected vibrations that are formed from the emitted fluid vibrations contacting and deflecting from surfaces of the mouth, such as teeth, dental or orthodontic fixtures, crowns, enamel, gums, roots, tongue, nerves, and the like. In one embodiment, the vibration emitter can be a sound emitter, such as a speaker, and the sensor receiver can be a sound receiver, such as a microphone.

However, in one aspect, the invention specifically excludes sound speakers and microphones that operate within the normal hearing range. It is preferred in some embodiments to use a sound that is outside of normal hearing ranges, such as being of a frequency and/or intensity that is outside of the normal hearing range. However, in one aspect, it can be desirable for the sound to be in the audible range, but outside of the normal speech and music ranges. In other aspects, it may be pleasant for the sound to be within the music range or speech range. Selecting any of these sound ranges, various implementations of the technology may be implemented, such as by allowing for pleasant auditory signals for imaging purposes to enhance patient compliance. Enhanced patient compliance can be beneficial to the patient of a dental procedure having their mouth imaged. The sound emitter can also be configured to allow for patient controlled and/or proceduralist controlled sound to that the patient or proceduralist can tune the sound to a desirable and/or pleasant sound, or to tune the sound to outside a perceptible range. The computing system 40 may include a signal filter configured to filter out environmental signals out of the received signal data, such as to eliminate sound signals not generated by respective signal generators/transmitters.

In one embodiment, the dental imaging system 10 may be configured as a vibrating system, which can use any type of vibration transmitter for vibration signal emitting and deflected vibration signal reception. The vibration system can include any types of vibrating devices that vibrate in a vibration frequency range that can be deflected from a surface of a various hardness and/or softness, and such deflected vibrations can be received, measured, and recorded.

In addition, the dental imaging system 10 may produce three-dimensional (i.e., 3D) images using one or more signal generators 20 (a.k.a. signal transmitters) and two or more signal receivers 30, i.e. a plurality of transmitters and receivers. A transmitter and receiver combination may be positioned within a common housing (i.e. of unitary construction) such that, together the combination is referred to as a transceiver 32. The transmitter emits a signal into a patient's mouth which then reflects or deflects the in-bound signal where it is received by one or more receivers in formats that are understood by a computing system 40. Further, the computing system 40 may be programmed to generate an accurate 3D model of the mouth and surfaces thereof. The computing system 40 may be programmed to manipulate the data so that the surfaces of the objects impacted by the transmitted signals can be determined. For surfaces of structures such as teeth, gums, tongue, cheeks or other structures (e.g., braces), the signal may be able to at least partially penetrate a certain distance, which allows for a combined external to internal imaging functionality and allows for more precise and accurate mouth 3D model generation.

In an exemplary use, the dental imaging system 10 can be used for imaging a mouth for many applications with a non-radiographic signal. Preferably, sound or vibration signals are sent and received as described above. In other words, the sound or vibration signal can be transmitted through or from a hand piece (e.g. a glove, wand, or the like) into a patients' mouth so that the signal interacts with (e.g., deflects from) the surfaces and some under-surface features in order to provide the receiver a deflected signal that can be converted to “signal data” for generating a real time 3D model of the mouth. More particularly, the signal can be transmitted into the mouth and optionally directed toward any surface in the mouth, whether natural like a crown or non-natural like an implant. Specifically, the signal and/or deflected signal can then be picked up (i.e. received) by the signal receiver 30 and converted to signal data. The signal data can be provided to a computing system 40 and processed by programming and data structures stored in non-volatile memory into a 3D model of the mouth and contents thereof. The dental imaging system 10 can also create 2D (two dimensional) slices of the 3D model in any plane relative to any axis, which 2D plane can be customized and modulated and changed in real time with the real time 3D or 2D model image being generated. It is understood that the computing system 40 is programmed to determine a difference between cheeks, teeth, gums, bone, tongue, dental fixtures, and the like in a mouth.

Further, the computing system 10 is programmed to generate a screen image (e.g. of a 3D model of a mouth) to be displayed on a graphical user interface (“GUI”), such as a computer display screen 50, touch screen, or the like. The 3D image of the mouth can be provided to a user, and the user can manipulate the image to show desirable features by implementing commands (i.e. inputs) into a user input interface. More particularly, the computing system 40 may include a real time image generator module 42 configured to receive the 3D model data generated generate images or video in real time based on received signal data. The image generator module 42 may be programmed to impart color or graphical indicia to graphical representations of cheeks, teeth, gums, bone, tongue, dental fixtures in the mouth, and the like. It is understood that the display screen 50 or similar visual display may be displaced or remote from the generators 20 and receivers 30 or, as described below, displaced or remote from a glove 60.

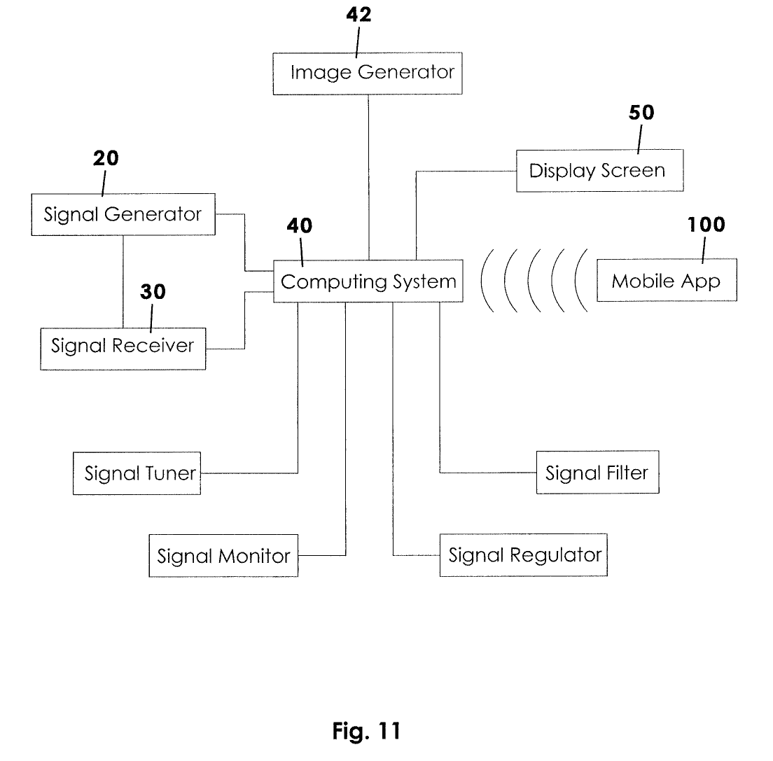

With further reference to the computing system 40, the signal generator 20 and signal receiver 30 can be operated or controlled by a computing system 40, such as a personal computer, workstation, server, mobile phone, or connected to a central computer or network. More particularly, the signal receiver 30 and data processing of the signal data can be performed by the computing system 40. The signal generators 20 and signal receivers 30 may be electrically connected to and in data communication with the computing system 40 via wires or wirelessly. In other words, data can be through a wire or wireless, e.g. Bluetooth. Optical data transmission is also possible. FIG. 11 illustrates the electronic components of an exemplary system.

In an embodiment, the system may include sensor and transmitter pads (not shown) that can be utilized in the mouth (e.g., intra-orally) or outside of the mouth (e.g., extra-orally) that transmit the sound or vibration signal into the mouth and into the features, such as tooth, gum, cheeks, bone, tongue, or the like, which signal is deflected and is received by a corresponding sensor and receiver. The signal is processed in real time to provide a near real time image on a display to the user. In some instances, a 3D screen can be used so that the user can see the model in 3D. In some instances, a ventricular 3D system can be used so that the user can view the 3D image without special glasses. In other instances, the 3D system can include 3D glasses for the user to see the 3D image in real time. The 3D can be any 3D technology that is known or developed whether the 3D technology uses 3D glasses or not.

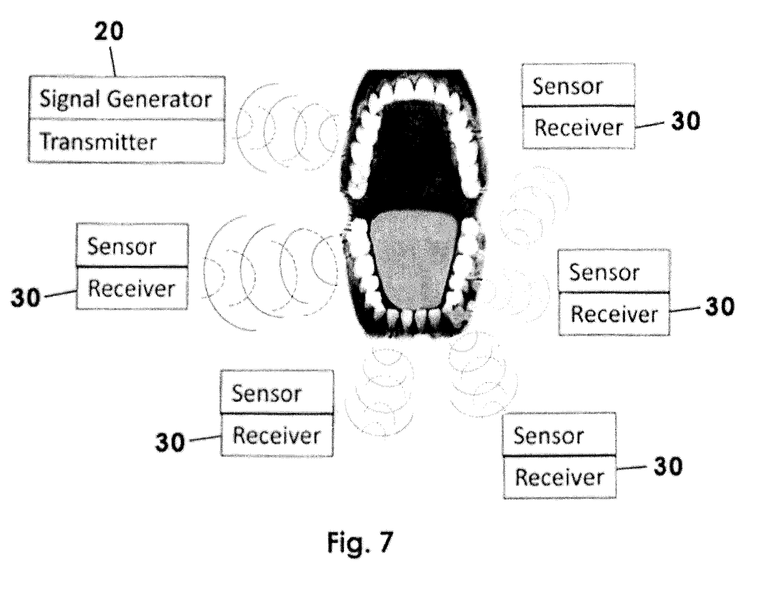

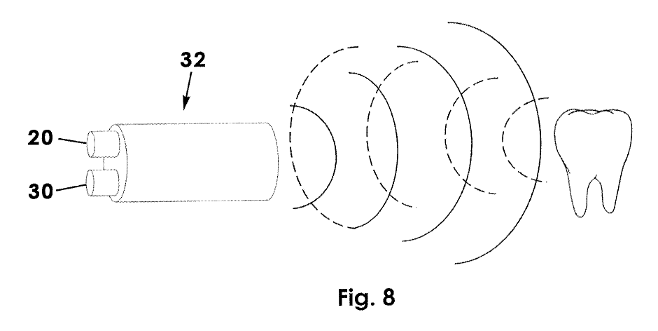

FIGS. 7 and 8 illustrate a signal generator/transmitter combination emitting one or more signals and one or more sensor/receiver combinations receiving the one or more signals or deflected signals thereof. It is also understood that a generator/transmitter 20 and receiver 30 may be constructed in a unitary housing and be referred to as a transceiver 32. Any transmitter or receiver described herein can be a transceiver, and can send and/or receive data, and include a processor for processing the data as well as process instructions that cause the transceiver 32 to operate. This can also include operating a signal generator or sensor associated with the transceiver.

The signals transmitted by a signal generator 20 interact with a surface or structure or feature or object in the mouth and are then received by the signal receivers 30 upon reflection or deflection therefrom so that the 3D model can be generated by the computing system 40. Any instruments or dental fixtures in the mouth will also interact with the signals and show up in the 3D model. This provides a real time and accurate 3D model of the mouth showing void space (air), liquid (saliva), tooth, gum tissue, bones, or dental devices or instruments. This allows the user to see the procedure via a generated 3D model image.

FIG. 1 shows a device that has a hand piece (described below to be a glove) that sends out a signal through a driver that can interact with teeth or implants and be received by a receiver pad (not shown). The received signal data of the mouth can then be sent to the 3D model generator and imaging component. The hand piece is capable of transmitting a signal into a mouth where it is reflected or deflected to a signal receiver 30, after which the received signal is delivered to computing system 40—specifically, to the 3D model generator. The signal may also go at least partially into the teeth, gums, jaw bones, tongue, cheeks, dental tools, or the like for imaging everything in the mouth.

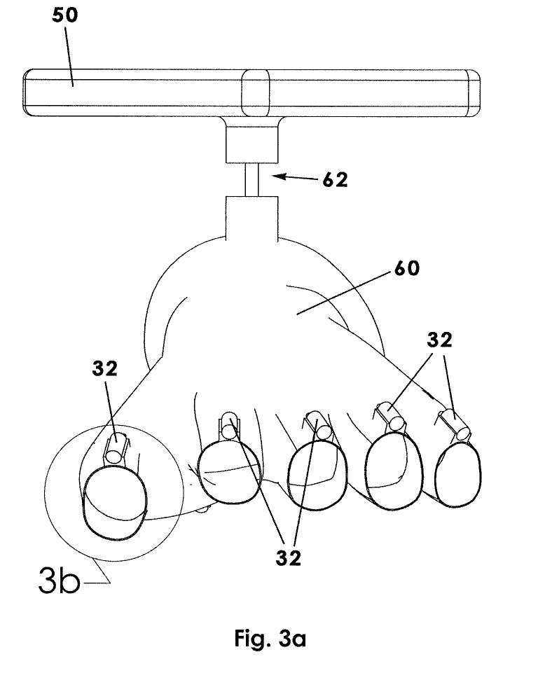

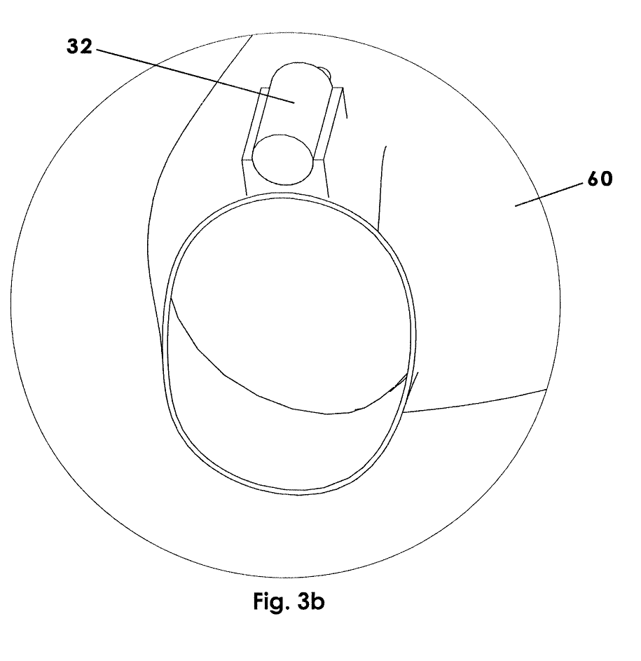

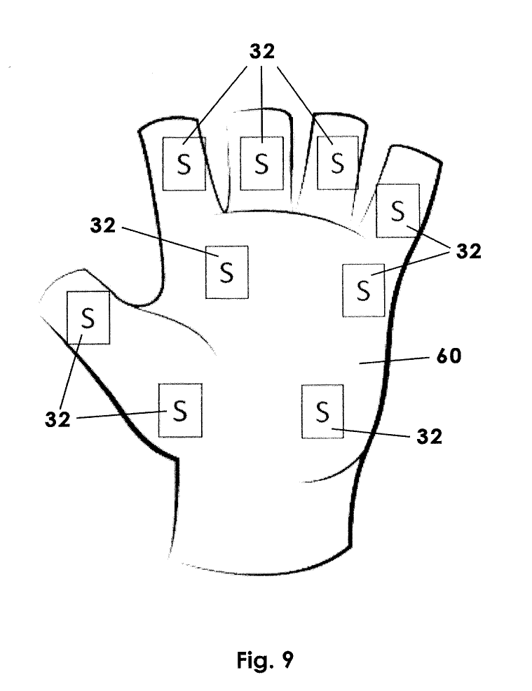

Now, more particularly, the present invention may include a glove 60 that may be donned by a user, such as a dentist, for use in imaging the mouth of a patient as described above and shown in FIGS. 1 to 6. The one or a plurality of signal transmitters 20 and one or a plurality of signal receivers 30 (i.e. transceivers) may be coupled to various positions on the glove 60 in a spaced apart arrangement (FIG. 9) (e.g., with “S” being any signal generator, transmitter and/or receiver). The glove 60 includes one or more signal transmitters and one or more signal receivers, which can be combined into one or more transceivers. The glove can include the sensors at selected positions, such as at the finger tips, or on the edges of the finger openings when the gloves are fingerless, see FIG. 3b. The transceivers may be positioned on either the front, back, or palm of the glove. FIG. 1 shows a sleeve embodiment that has a display mount 62 to which a display screen 50 may be adjustably mounted so that an angle of the screen can be optimized for the user to work with their hands and image a mouth.

Similarly, the present invention may include a probe 60a that may be held by a user (e.g. a dentist) and positioned in proximity to a patient's mouth for use in imaging the mouth of a patient as described above and shown in FIGS. 1 to 6. Alternatively, the probe 60a may be held by a stand or other framework so as to remain stable and fixed during an entire dental procedure. It is understood that the probe 60a may define an open interior area and include a plurality of signal transceivers 32 in a spaced apart and functional manner as described above (not shown).

In another aspect, the dental imaging system 10 may include a software application 100 running on a mobile device separate and remote from the glove 60, respective signal generators 20 and signal receivers 30, computing system 40, and display 50. The software application may include a graphic user interface (GUI), input controls, and its own dedicated display screen, the software application being configured to operate the entire system described above.

In use, a dentist or other user may position the single or plurality of signal generators 20 (transmitters) proximate the mouth of a patient and then actuate the signal transmission as described above. The signals may include sound or vibration signals that reflect and deflect off of the teeth, dental fixtures, cheeks, gums, etc. and are received by respective signal receivers 30, converted to signal data and delivered to an associated computing system 40. The computing system 40 may include programming instructions and data stored in memory capable of generating a 3D model (e.g. of the patient's mouth) based on the signal data and predetermined data on the relative teeth and structures being imaged. The 3D images or video may be updated continuously and displayed in real time on a display screen 50.

Accordingly, a dentist or surgeon conducting a dental procedure, e.g. embedding tooth inserts, is able to visualize all of the teeth and mouth structures of a patient in real time so that the surgical procedure may be executed with precision, speed, and confidence.

It is understood that while certain forms of this invention have been illustrated and described, it is not limited thereto except insofar as such limitations are included in the following claims and allowable functional equivalents thereof.

1. A dental imaging system for imaging a mouth in real time, comprising:
a signal generator that selectively transmits one or more non-radiological signals into the mouth;
a signal receiver that selectively receives the one or more non-radiological signals reflected from the mouth and generates signal data therewith;
a computing system in data communication with the signal generator and in data communication with the signal receiver so as to receive signal data from the signal receiver, the computing system being configured to process the received signal data to generate in real time a 3-dimensional model (“3D model”), the 3D model having a 3D model visualization component;
wherein the computing system includes a real time image generator module programmed to generate images or video in real time according to the received signal data; and
a display screen in data communication with the computing system that is configured to display the 3D visualization component of the 3D model in real time.

2. The dental imaging system as in claim 1, wherein:
the signal generator is a plurality of signal generators, each signal generator being spaced apart from an adjacent signal generator;
the signal receiver is a plurality of signal receivers, each signal receiver being spaced apart from an adjacent signal receiver.

3. The dental imaging system as in claim 2, wherein:
the plurality of signal generators and the plurality of signal receivers form a plurality of signal transceivers, each signal transceiver having a respective signal generator and a respective signal receiver integrated in a unitary construction;
each signal transceiver is spaced apart from an adjacent signal transceiver such that the received signal data from said plurality of signal receivers is processed simultaneously by the computing system to generate the 3D model.

4. The dental imaging system as in claim 1, wherein the signal generator and the signal receiver are combined to form a signal transceiver having a unitary construction.

5. The dental imaging system as in claim 3, wherein the computing system includes a signal filter that filters environmental signals out of said received signal data, the environmental signals include sound signals not generated by respective signal transmitters.

6. The dental imaging system as in claim 1, wherein the signal generator generates and emits vibration signals and the signal receiver receives the emitted vibration signals, said computing system configured to process said received vibration signals.

7. The dental imaging system as in claim 1, wherein the signal generator generates and emits sound signals into the mouth and each signal receiver receives the emitted sound signals, said computing system configured to convert said received sound signals to signal data for generating a real-time 3D model.

8. (canceled)

9. (canceled)

10. The dental imaging system as in claim 1, wherein the emitted signal of the signal generator is configured to partially penetrate and reflect from a natural or unnatural object in the mouth.

11. The dental imaging system as in claim 3, wherein said computing system is programmed to determine, based on the received signal data, a difference between cheeks, teeth, gums, bone, and tongue within a mouth.

12. The dental imaging system as in claim 10, wherein the computing system is programmed to impart color or graphical indicia on the 3D model indicative of cheeks, teeth, gums, bone, and tongue within a mouth based on the received signal data.

13. The dental imaging system as in claim 1, further comprising a glove wearable on a person's hand, the signal generator and the signal receiver being mounted on the glove.

14. The dental imaging system as in claim 3, further comprising a glove wearable on a person's hand, wherein the plurality of transceivers are mounted to the glove and spaced apart from one another in predetermined positions in data communication with the computing system.

15. The dental imaging system as in claim 12, wherein the glove is fingerless.

16. The dental imaging system as in claim 12, wherein the glove includes a display mount and the display screen is coupled to the display mount.

17. The dental imaging system as in claim 12, wherein the computing system and the display screen are displaced from the glove.

18. The dental imaging system as in claim 1, wherein the display screen is taken from a group comprising a 3D image display, a 2D image display, and a touch screen.

19. The dental imaging system as in claim 2, wherein the computing system is a smart phone in data communications with said plurality of transceivers using Bluetooth or wifi wireless protocols.

20. The dental imaging system as in claim 2, further comprising a software application executing on a remote computing device in wireless data communication with the computing system, the software application having programming for controlling the computing system remotely.

21. (canceled)

22. A method for imaging a mouth in real time during a dental procedure, comprising:
actuating a signal generator to selectively transmit one or more non-radiological signals into the mouth;
actuating a signal receiver to selectively receive the one or more transmitted non-radiological signals reflected from the mouth;
a computing system processing the received signal data and generating a 3-dimensional model (“3D model”) in real time, the 3D model having a 3D model visualization component;
the computing system having a real time image generator module generating images or video in real time according to the received signal data; and
displaying the 3D visualization component of the 3D model on a display screen proximate the signal generator and the signal receiver.

23. The dental imaging method as in claim 22, wherein:
the signal generator includes a plurality of signal generators, each signal generator being spaced apart from an adjacent signal generator;
the signal receiver includes a plurality of signal receivers, each signal receiver being spaced apart from an adjacent signal receiver.

24. The dental imaging method as in claim 22, wherein:
the plurality of signal generators and the plurality of signal receivers form a plurality of signal transceivers, each signal transceiver having a respective signal generator and a respective signal receiver integrated in a unitary construction;
each signal transceiver is spaced apart from an adjacent signal transceiver such that the received signal data from said plurality of signal receivers is processed simultaneously by the computing system to generate the 3D model.

25. The dental imaging method as in claim 22, wherein each signal generator generates and emits vibration signals and each signal receiver receives the emitted vibration signals, said computing system configured to process said received vibration signals.

26. A method for imaging a mouth in real time during a dental procedure, comprising:
actuating a signal generator to selectively transmit one or more non-radiological signals into the mouth;
actuating a signal receiver to selectively receive the one or more transmitted non-radiological signals reflected from the mouth;
a computing system processing the received signal data and generating a 3-dimensional model (“3D model”), the 3D model having a 3D model visualization component;
displaying the 3D visualization component of the 3D model on a display screen proximate the signal generator and the signal receiver;
wherein said signal generator generating and emitting sound signals into the mouth and each signal receiver receiving the emitted sound signals and converting said received signal into signal data for generating a real-time 3D model;
wherein said computing system determining, based on the received signal data, a difference between cheeks, teeth, gums, bone, and tongue within a mouth.

27. The dental imaging method as in claim 22, further comprising generating images or video in real time according to the received signal data via a real time image generator module of the computing system.

28. The dental imaging method as in claim 22, further comprising the emitted signal of the signal generator penetrating and reflecting from a natural or unnatural object in the mouth.

29. (canceled)

30. (canceled)

31. (canceled)

32. The dental imaging method as in claim 23, further comprising donning a glove wearable on a person's hand, wherein the plurality of transceivers are mounted to the glove and spaced apart from one another in predetermined positions in data communication with the computing system.

33. (canceled)

34. (canceled)

35. (canceled)
